(19) United States
(12) Patent Application Publication
(10) Pub. No.: US 2004/0186549 A1
(43) Pub. Date: Sep. 23, 2004

(54) Braided stent with looped ends and method for making same

(76) Inventor: Swaminathan Jayaraman, Fremont, CA (US)

Correspondence Address: PAUL D. BIANCO: FLEIT, KAIN, GIBBONS,, GUTMAN, BONGINI, & BIANCO P.L., 601 BRICKELL KEY DRIVE, SUITE 404, MIAMI, FL, 33131, US

(21) Appl. No.: 10/391,826

(22) Filed: Mar. 19, 2003

(51) Int. Cl. A61F 2/06

(52) U.S. Cl. 623/1.15; 623/1.22; 623/23.7

(57) ABSTRACT

The present invention relates to an implantable stent having looped ends. The stent includes a plurality of wire strands braided into a tubular structure such that the ends of the plurality of wire strands are located at a first end of the tubular structure. The stent further includes a first plurality of loops located at the first end of the tubular structure, each loop formed from a pair of ends of the plurality of wire strands. The stent further includes a second plurality of loops located at a second end of the tubular structure, each loop formed from a length of the plurality of wire strands.

### FIELD OF THE INVENTION

The present invention relates to an implantable stent, and in particular to a braided stent with looped ends and a method for making the stent.

### BACKGROUND OF THE INVENTION

The use of stents has increased since their inception. Stents have changed the manner in which patients are treated, by providing a less invasive and less risky surgical procedure. In coronary care, for example, complications following angioplasty can result in acute damage to artery walls, which, prior to the development of stent technology, required immediate bypass surgery. Stents are now recognized as a viable means of avoiding such procedures, because implantation of such a mechanical device into the area of concern allows the artery walls to be reinforced with permanent artificial scaffolding. Additionally, stents are now recognized as an effective modality for reducing the frequency of restenosis, the recurrent narrowing of the lumen (cavity or channel within a body tube).

A stent is a cylindrically shaped device intended to act as a permanent prosthesis. A stent is deployed in a body lumen from a radially compressed configuration into a radially expanded configuration which allows it to contact and support the body lumen. The stent can be made to be radially self-expanding or expandable by the use of an expansion device. The self-expanding stent is made from a resilient springy material while the device expandable stent is made from a material which is plastically deformable. A plastically deformable stent can be implanted during a single angioplasty procedure by using a balloon catheter bearing a stent which has been crimped onto the balloon. Stents radially expand as the balloon is inflated, forcing the stent into contact with the body lumen thereby forming a supporting relationship with the vessel walls. Deployment is effected after the stent has been introduced percutaneously, transported transluminally and positioned at a desired location by means of the balloon catheter.

Stents designed for use in arteries are generally fabricated from metal wireforms or laser cut from metallic tubular shapes. Wireforms may be twisted or coiled using manual or automatic coil winding machines to make the stent, or interwoven using braiding or knitting technology. Individual or multiple wireforms may be used to create the stent. Prior art stents include metal-polymer composites, in which a metal layer is sandwiched between two layers of a polymer. Alternatively, the faces of the metal layer are coated using thin film deposition techniques, such as Pulsed Laser Deposition, which allow polymer films of micron-level thickness to be applied. The metal layer may also be sandwiched between two layers of biocompatible material.

Stents do not come without drawbacks, as a foreign object introduced into the body can produce undesirable results. Among the problems encountered with implantation are tearing or cracking of the artery lining. In addition, the stent may irritate the lumen, resulting in blood clot formation on the stent itself. Serious consequences may result, including the need for further invasive procedures and concomitant increased risks.

When inserted within an artery, an interwoven stent with loose wire ends may chafe and irritate the fragile epithelium tissue lining the inside diameter of the artery. In addition, when submitted to enough movement, loose wire ends can induce the unraveling of the stent. To prevent this, the wire strands forming the stent may be coated for protecting the artery from contact with the material of the wire strands, or a coating for releasing a pharmaceutical agent into the blood stream. However, delamination of the coating from the wires is possible since the ends of the wires may expose the coating/wire interface. Therefore, there is a need for an improved braided stent which reduces irritation of the interior wall of the artery and withstands structural deterioration over time.

### SUMMARY OF THE INVENTION

The present invention relates to an implantable stent formed of braided wire strands having looped ends so as to minimize the risk of fraying of the wire strands at the ends of the stent and minimize the risk of irritation of the artery. The stent includes a first plurality of loops located at the first end of the tubular structure. Each loop is formed from a pair of ends of the wire strands. The stent further includes a second plurality of loops located at a second end of the tubular structure. Each of these loops is formed from a length of the wire strands.

The present invention also relates to a method for making the described stent. The method for making stent includes the creation of loops on both ends of the stent such that the ends of the wire strands, comprising the stent, are not subject to fraying, unraveling or irritation of the artery in which it is implanted.

In one embodiment, the ends of the wire strands at the first end of the tubular structure are trimmed to be flush with the first plurality of loops. In another embodiment, the pair of ends of the wire strands that comprise each loop of the first plurality of loops are secured to each other. In yet another embodiment, the stent includes the wire strands braided into axially spaced apart helices concentric on a central axis of the stent.

The present invention further relates to a method of making an implantable stent having looped ends. The method includes forming a first plurality of loops from a plurality of wire strands. The method further includes arranging the first plurality of loops in a circle such that each of loops is equally spaced apart around the circumference of the circle, wherein the ends of the wire strands extend in the same axial direction perpendicular to the plane of the circle. The method further includes braiding the ends of the wire strands to form a tubular structure, wherein the ends of the wire strands are located at a first end of the tubular structure. The method further includes forming a second plurality of loops at the first end of the tubular structure. Each of these loops is formed from a pair of ends of the wire strands.

In one embodiment, the ends of the wire strands are trimmed to be flush with the second plurality of loops. In another embodiment, the pair of ends that comprise each of the second plurality of loops are secured together. In yet another embodiment, the braiding step further includes braiding the ends of the wire strands into axially spaced apart helices concentric on a central axis of the tubular structure.

### BRIEF DESCRIPTION OF THE DRAWINGS

Preferred features of the present invention are disclosed in the accompanying drawings, wherein similar reference characters denote similar elements throughout the several views, and wherein:

FIG. 1 shows a perspective view of a stent, in one embodiment of the present invention;

FIG. 2A shows an enlarged view of a first end of the stent of FIG. 1;

FIG. 2B shows an enlarged view of the process of creating one embodiment of a loop for the first end of the stent;

FIG. 2C shows an enlarged view of a loop for the first end of the stent, in the embodiment of FIG. 2B;

FIG. 2D shows an enlarged view of the process of creating another embodiment of a loop for the first end of the stent;

FIG. 2E shows an enlarged view of a loop for the first end of the stent, in the embodiment of FIG. 2D;

FIG. 2F is an electron microscope image of an example of a loop in the first end of the stent;

FIG. 3A shows an enlarged view of a second end of the stent of FIG. 1;

FIG. 3B shows an enlarged view of one embodiment of a loop for the second end of the stent;

FIG. 3C shows an enlarged view of another embodiment of a loop for the second end of the stent;

FIG. 3D is an electron microscope image of an example of a loop in the second end of the stent;

FIG. 4A shows an enlarged view of a middle section of the stent of FIG. 1;

FIG. 4B is an electron microscope image of an example of the middle section of the stent of FIG. 1;

FIG. 5 shows a single wire strand, bent during the process of making a stent in one embodiment of the present invention;

FIG. 6 shows the single wire strand of FIG. 5, further looped during the process of making a stent in one embodiment of the present invention;

FIG. 7 shows a plurality of wire strands, such as that of FIG. 6, looped and arranged together, during the process of making a stent in one embodiment of the present invention;

FIG. 8 shows the wire strands of FIG. 7, further braided during the process of making a stent in one embodiment of the present invention;

FIG. 9 is an enlarged view of the ends of the wire strands of FIG. 8, braided during the process of making a stent in one embodiment of the present invention;

FIG. 10 shows the ends of the wire strands of FIG. 9, further looped during the process of making a stent in one embodiment of the present invention;

FIG. 11 shows the ends of the wire strands of FIG. 10, further trimmed during the process of making a stent in one embodiment of the present invention;

FIG. 12 shows a top view of a tool used during the process of making a stent in one embodiment of the present invention;

FIG. 13 shows a perspective view of the tool of FIG. 12; and,

FIG. 14 shows a perspective view of the tool of FIG. 12 used during the process of making a stent in one embodiment of the present invention.

### DETAILED DESCRIPTION OF THE EMBODIMENTS

For convenience, the same or equivalent elements in the various embodiments of the invention illustrated in the drawings have been identified with the same reference numerals. Further, in the description that follows, any reference to either orientation or direction is intended primarily for the convenience of description and is not intended in any way to limit the scope of the present invention thereto. Finally, any reference to a particular biological application, such as use of a stent for cardiovascular applications, is simply used for convenience as one example of a possible use for the invention and is not intended to limit the scope of the present invention thereto.

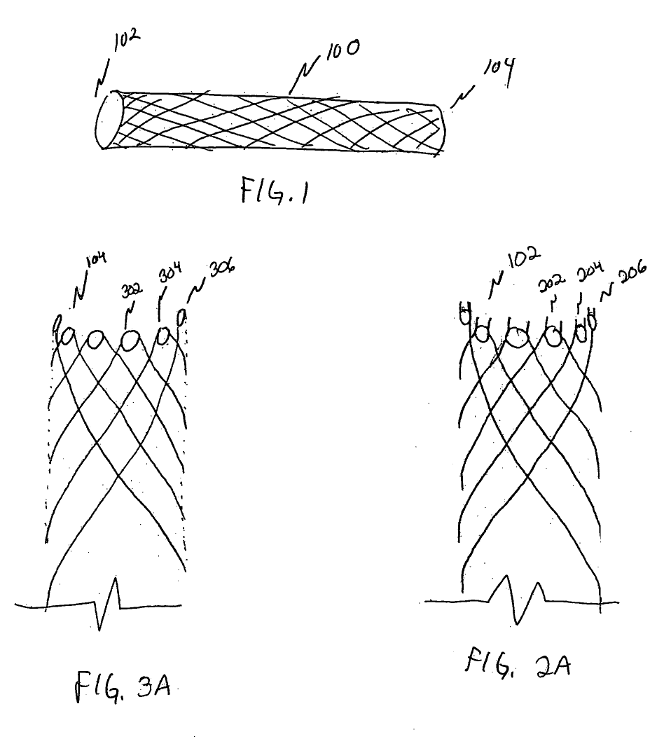

FIG. 1 shows a perspective view of a stent 100, in one embodiment of the present invention. The figure shows that the stent 100 is a tubular structure comprising a first end 102 and a second end 104. The stent 100 is a braided stent comprised of a plurality of wire strands, described in greater detail below. The stent 100 is braided or woven in a cross-meshed manner, whereby the plurality of wire strands are braided into axially spaced apart helices concentric on a central axis of the tubular structure. It should be noted that although FIG. 1 shows a particular manner of braiding or weaving the plurality of wire strands, the present invention supports other ways of braiding or weaving the plurality of wire strands. The wire strands can be coated with a coating (such as a polymeric coating, either biodegradable or not, and with or without a therapeutic agent), either before or after forming stent 100. For example, the stent of the present invention may be formed by encapsulated stent preforms. Encapsulated stent preforms are disclosed in U.S. Pat. No. 9,475,235 entitled, “Encapsulated Stent Preform.” The disclosure of that patent is incorporated hereby by reference.

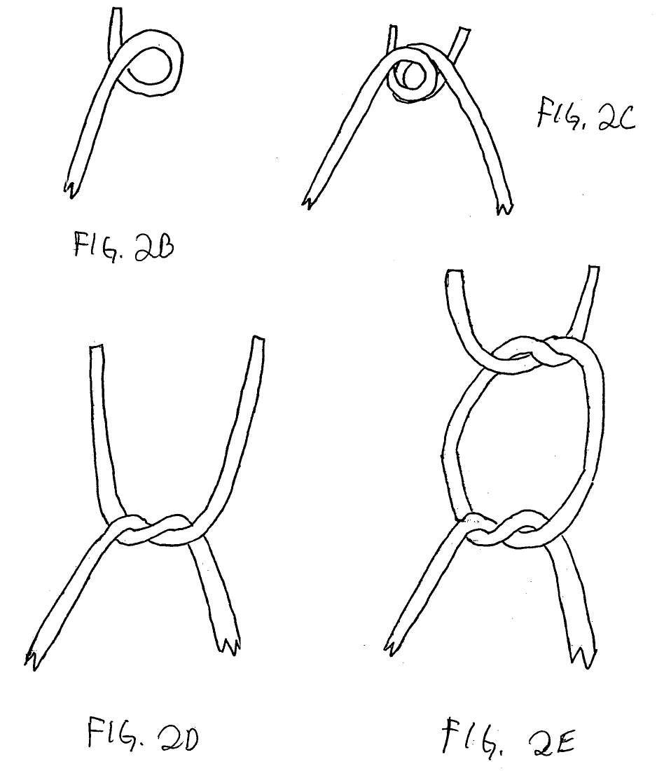

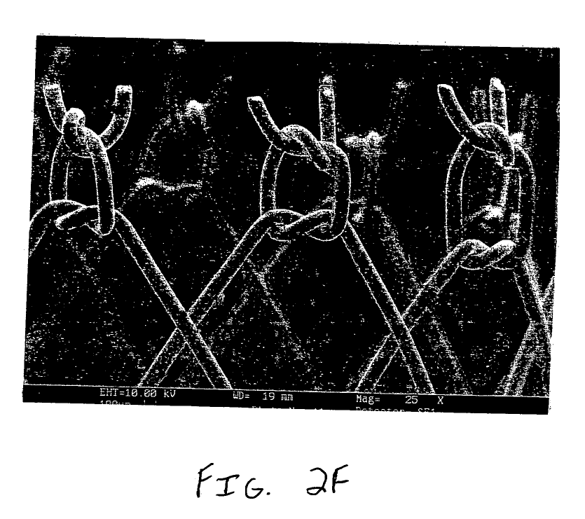

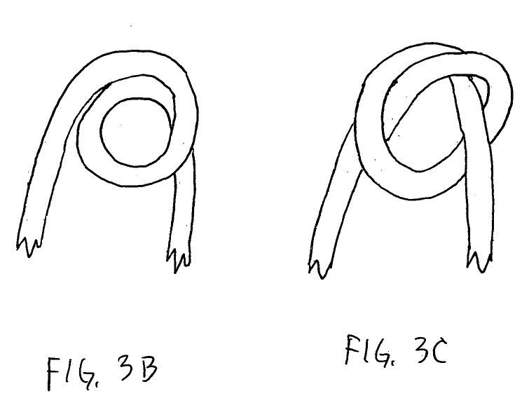

FIG. 2A shows an enlarged view of the first end 102 of the stent 100 of FIG. 1. FIG. 2A is a side view of the first end 102 of the stent 100, showing only a portion of the tubular stent 100. It is noted that the first end 102 of stent 100 is a circular end of a tubular structure, though only a portion of the first end 102 is shown for illustrative purposes.

The first end 102 of stent 100 contains all of the ends of the plurality of wire strands. That is, the first end 102 of stent 100 is comprised solely of ends of the wire strands. Note that an end is a length of a wire strand including an end-point of the wire strand.

FIG. 2A also shows looped ends 202, 204 and 206, among others, included in the first end 102 of the stent 100. The looped ends 202, 204 and 206 are formed from ends of the wire strands. The presence of all of the ends of the wire strands in the first end 102 of the stent 100 is attributed to the method in which the stent 100 is braided or woven from the plurality of wire strands. The method of braiding the stent 100 from the plurality of wire strands is described in greater detail below.

FIGS. 2B and 2C show an enlarged view of the process of creating one embodiment of a loop for the first end 102 of the stent 100. Note that the loops in the first end 102 of the stent 100 include two ends of the plurality of wire strands. FIG. 2B shows a loop created in an end of one wire strand, comprising a simple loop rotating about 360 degrees. At no point does an end of the wire strand travel through the loop made in the end of the wire strand. Next, in FIG. 2C, two loops, such as the one described in FIG. 2B are arranged one on top of the other. That is, the loop of FIG. 2B and a second similar loop (reversed in perspective) are arranged such that the orifices created by their respective loops are aligned.

FIGS. 2D, 2E and 2F show an enlarged view of the process of creating another embodiment of a loop for the first end 102 of the stent 100. Note that the loops in the first end 102 of the stent 100 include two ends of the plurality of wire strands. FIG. 2D shows two ends of the wire strands creating an overhand knot. Next, in FIG. 2E, the ends of the wire strands resulting from the overhand knot of FIG. 2D are further used to create another overhand knot spaced apart from the first overhand knot to create a loop. FIG. 2F is an electron microscope image of an example of a loop in the first end 102 of the stent 100, consistent with the loop of FIG. 2E.

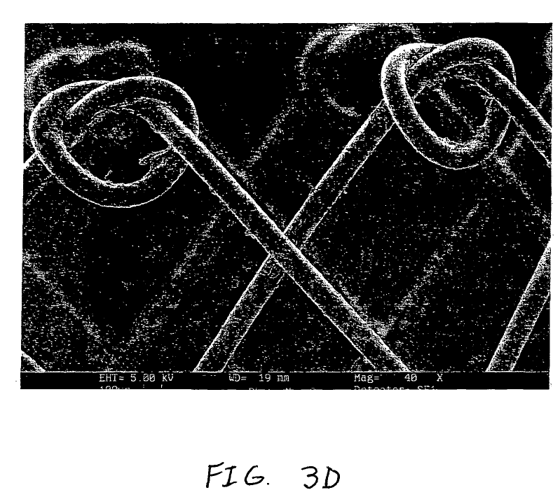

FIG. 3A shows an enlarged view of the second end 104 of the stent 100 of FIG. 1. FIG. 3A is a side view of the second end 104 of the stent 100, showing only a portion of the tubular stent 100. It is noted that the second end 104 of stent 100 is a circular end of a tubular structure, though only a portion of the second end 104 is shown for illustrative purposes.

The second end 104 of stent 100 does not contain any ends of the wire strands. That is, the second end 104 of stent 100 is comprised solely of bights of the plurality of wire strands. A bight is a length of a mid-section of a wire strand, not including an end of the wire strand. The ends of the wire strands are described in greater detail below. An end is a length of a wire strand including an end-point of the wire strand.

FIG. 3A also shows looped ends 302, 304 and 306, among others, included in the second end 104 of the stent 100. The looped ends 302, 304 and 306 are composed of bights of the wire strands and do not contain ends of the wire strands. The lack of ends of the wire strands in the second end 104 of the stent 100 is attributed to the method in which the stent 100 is braided or woven from the plurality of wire strands. The method of braiding the stent 100 from the plurality of wire strands is described in greater detail below.

FIG. 3B shows an enlarged view of one embodiment of a loop for the second end 104 of the stent. FIG. 3B shows a loop created on a bight of a wire strand, comprising a simple loop rotating approximately 540 degrees. At no point does an end of the wire strand travel through the loop made in the bight of the wire strand.

FIGS. 3C and 3D show an enlarged view of another embodiment of a loop for the second end 104 of the stent 100. FIG. 3C shows a loop created on a bight of a wire strand formed by a simple overhand knot. In creating the loop of FIG. 3C, the wire strand rotates approximately 540 degrees and the end of the wire strand travels through the loop made in the bight of the wire strand. FIG. 3D is an electron microscope image of an example of a loop in the second end 104 of the stent 100, consistent with the loop of FIG. 3C.

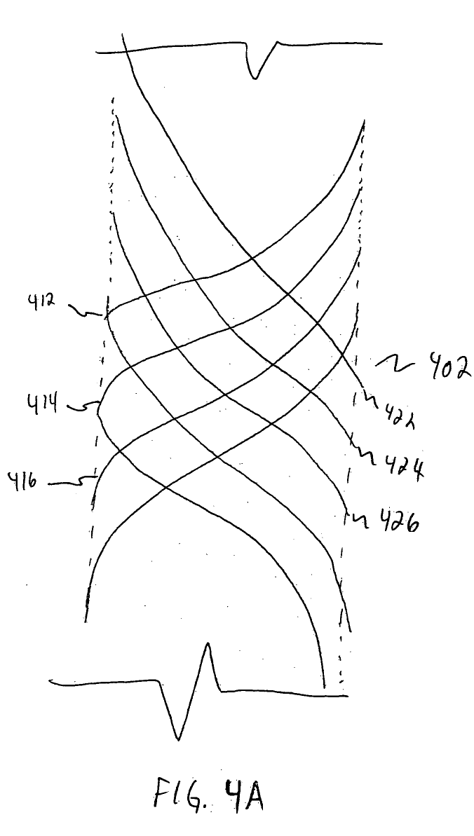

FIG. 4A shows an enlarged view of a middle section 402 of the stent 100 of FIG. 1. FIG. 4A is a side view of a middle section 402 of the stent 100 and shows only a portion of the middle section 402 of the tubular stent 100. It is noted that the middle section 402 of stent 100 is a circular section of a tubular structure, though only a portion of the middle section 402 is shown for illustrative purposes.

The stent 100 is braided or woven in a cross-meshed manner, whereby the plurality of wire strands are braided into axially spaced apart helices concentric on a central axis of the tubular structure. As explained above, although FIG. 4A shows a particular manner of braiding or weaving the plurality of wire strands, the present invention supports other ways of braiding the wire strands.

FIG. 4A also shows portions of wire strands 412, 414 and 416, among others. Note that the portions of wire strands 412, 414 and 416 run parallel to each other, and as the wire strands are braided around the tubular structure of the stent 100, the portions of wire strands 412, 414 and 416 do not intersect each other. Also note that the portions of wire strands 422, 424 and 426 run parallel to each other, and as the wire strands are braided around the tubular structure of the stent 100, the portions of wire strands 422, 424 and 426 do not intersect each other. As shown in the figure, the portions of wire strands 412, 414 and 416 intersect with portions of wire strands 422, 424 and 426 as the wire strands are braided around the tubular structure of the stent 100. In doing so, the present method of braiding creates diamond shaped openings in the tubular structure of the stent 100. Note that although FIG. 4A shows only portions of wire strands, 412, 414, 416, 422, 424 and 426, the stent 100 comprises additional wire strands (not shown) necessary for braiding or weaving a fully tubular structure.

FIG. 4B is an electron microscope image of an example of the middle section 402 of the stent 100, consistent with the middle section shown in FIG. 4A.

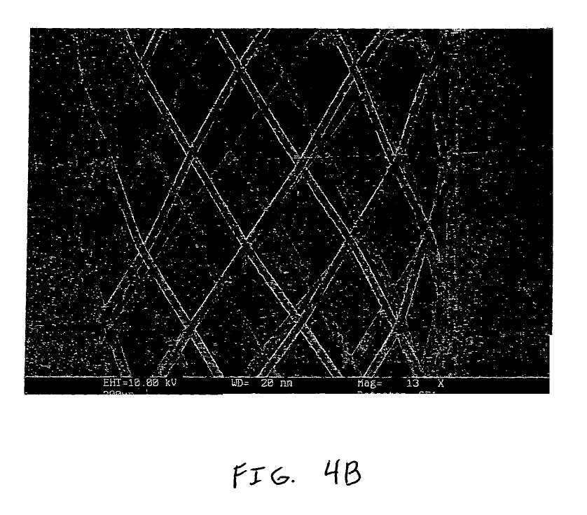

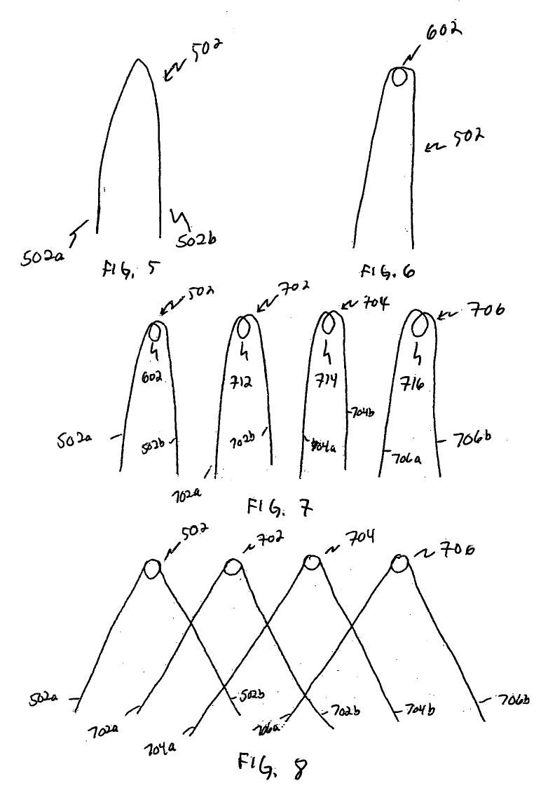

In chronological sequence, FIGS. 5, 6, 7 and 8 describe the formation of the second end 104 of stent 100, while FIGS. 9, 10 and 11 describe the formation of the first end 102 of stent 100. In an embodiment of the present invention, the method of making the stent 100 begins with a plurality of wire strands, which are ultimately braided or woven together to form a tubular structure. The stent 100 is braided such that the second end 104 is formed first, the middle section 402 is formed next and the first end 102 is formed last.

FIG. 5 shows a single wire strand 502 bent during the process of making a stent in one embodiment of the present invention. The first step in the method of making the stent 100 includes the bending of at least one wire strand, forming a length of bight of the wire strand 502 and two ends 502a and 502b.

FIG. 6 shows the single wire strand 502 of FIG. 5, further bent to create a loop 602. Preferably, a loop at the second end 104 of the stent 100 is a bend in the wire strand 502 which turns approximately 540 degrees. However, the loop may also be formed from bends which turn in the range of approximately 90 degrees to approximately 900 degrees.

Subsequently, the wire strand 502 is arranged in proximity to a plurality of other wire strands 702, 704 and 706, all of which have been bent and looped. Specifically, the wire strands 502, 702, 704 and 706 are arranged in a circle, such that the loops of the wire strands are equally spaced apart around the circumference of the circle. FIG. 7 shows the wire strands 502, 702, 704 and 706 looped and arranged together, during the process of making a stent in one embodiment of the present invention. FIG. 7 is a side view of the circle around which the plurality of wire strands are arranged, showing only a portion of the circle. It is noted that the wire strands are arranged around the entire circumference of the circle, though only a portion of the circle is shown for illustrative purposes.

FIG. 7 also shows the wire strands 502, 702, 704 and 706 arranged together. The wire strands 702, 704, 706 have been bent and looped in the same way that wire strand 502 has been bent and looped. However, each wire strand may be bent and looped differently if desired. Wire strand 502 includes a loop 602 and two ends 502a and 502b. Wire strand 702 includes a loop 712 and two ends 702a and 702b. Likewise, wire strand 704 includes a loop 714 and two ends 704a and 704b, and wire strand 706 includes a loop 716 and two ends 706a and 706b.

FIG. 8 shows the plurality of wire strands 502, 702, 704 and 706 braided during the process of making a stent in one embodiment of the present invention. The ends 502a and 502b, 702a and 702b, 704a and 704b, 706a and 706b of the wire strands 502, 702, 704 and 706, respectively, are braided or woven together in a cross mesh manner, described in more detail with reference to FIG. 4A and FIG. 4B. Like FIG. 7, FIG. 8 is a side view of the circle around which the wire strands are arranged, showing only a portion of the circle. It is noted that the wire strands are arranged around the entire circumference of the circle, though only a portion of the circle is shown for illustrative purposes.

Stent 100 is braided or woven in a cross-meshed manner, whereby the wire strands 502, 702, 704, 706 are braided into axially spaced apart helices concentric on a central axis of the tubular structure. As explained above, although FIG. 8 shows a particular manner of braiding the wire strands 502, 702, 704 and 706, the present invention supports other ways of braiding the plurality of wire strands.

Note in FIG. 8 that ends 502a, 702a, 704a and 706a of the wire strands 502, 702, 704 and 706 run parallel to each other, and as the wire strands are braided around the tubular structure of the stent 100, the ends 502a, 702a, 704a and 706a do not intersect. Also note that the ends 502b, 702b, 704b and 706b of the wire strands 502, 702, 704 and 706 run parallel to each other, and as the wire strands are braided around the tubular structure of the stent 100, the ends 502b, 702b, 704b and 706b do not intersect. As shown in the figure, the ends 502a, 702a, 704a and 706a of the wire strands 502, 702, 704 and 706 intersect with the ends 502b, 702b, 704b and 706b of the wire strands 502, 702, 704 and 706 as the wire strands are braided around the tubular structure of the stent 100. In doing so, the present method of braiding creates diamond shaped openings in the tubular structure of the stent 100. The ends 502a, 502b, 702a, 702b, 704a, 704b, 706a and 706b of the plurality of wire strands 502, 702, 704, 706 are braided until the desired length of the stent 100 is attained.

FIG. 9 is an enlarged view of the ends of the wire strands braided during the process of making a stent in one embodiment of the present invention. FIG. 9 shows the first end 102 of the stent 100 as the process of braiding progresses. FIG. 9 is a side view of the first end 102 of the stent 100, showing only a portion of the tubular stent 100. It is noted that the first end 102 of stent 100 is a circular end of a tubular structure, though only a portion of the first end 102 is shown for illustrative purposes.

The ends 502a, 502b, 702a, 702b, 704a, 704b, 706a and 706b of the wire strands 502, 702, 704 and 706 are braided during the process of making the stent 100. Upon attainment of the appropriate length of the stent 100, these ends form the first end 102 of the stent 100. Then, the ends 502a, 502b, 702a, 702b, 704a, 704b, 706a and 706b are bent to form a plurality of loops at the first end 102 of the stent 100. Preferably, a loop at the first end 102 of the stent 100 is formed by using two ends of the wire strands and by creating two spaced-apart, simple overhand knots. However, the loops may also be formed from bends in the wire sections which turn in the range of approximately 90 degrees to approximately 900 degrees.

Although FIG. 9 shows two ends from the same wire strand being formed into a loop, each end 502a, 502b, 702a, 702b, 704a, 704b, 706a and 706b may be looped with an end from a different wire strand. For example, end 502a is shown paired end 502b to form a loop. However, end 502a may be looped with ends 702b, 704b, or 706b.

FIGS. 10 and 11 show that a plurality of loops 1002, 1004, 1006 and 1008 are created, each loop formed from a pair of ends of the wire strands. The ends 502a, 502b, 702a, 702b, 704a, 704b, 706a and 706b are not flush with the plurality of loops 1002, 1004, 1006 and 1008. In order to reduce the risk of fraying or irritation of the artery into which the stent 100 is implanted, the ends of the wire strands are clipped so they are flush with the plurality of loops. FIG. 11 shows the ends 502a, 502b, 702a, 702b, 704a, 704b, 706a and 706b of the wire strands trimmed to be flush with the plurality of loops 1002, 1004, 1006 and 1008.

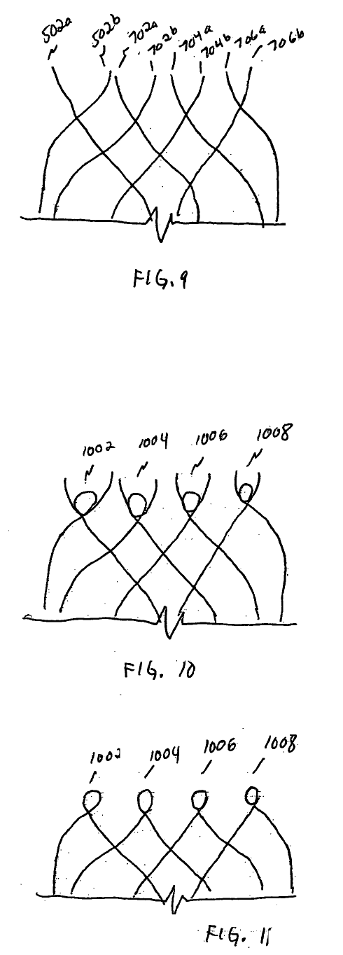

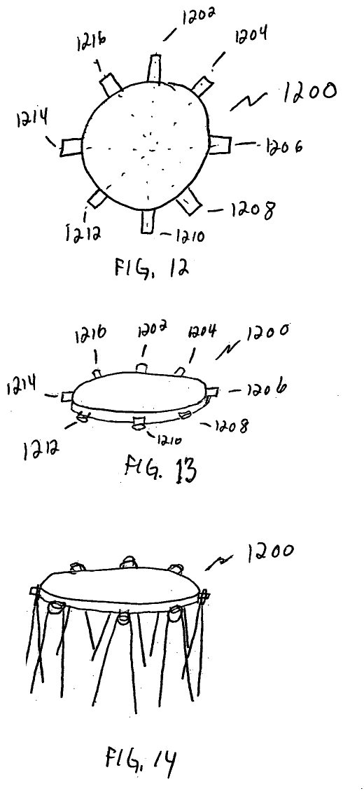

FIG. 12 shows a top view of a tool 1200 used during the process of making the stent 100 in one embodiment of the present invention. The tool 1200 is a circular disc substantially the same diameter as the desired inner diameter of the stent 100. The tool 1200 also includes a group of cylindrical projections 1202, 1204, 1206, 1208, 1210, 1212, 1214 and 1216 evenly spaced around the circumference of the circular disc of tool 1220. Each of the cylindrical projections is coupled to the outside circumference of the tool 1220 such that the axis of each cylindrical projection is aligned radially out from the center-point of the circular tool 1200.

FIG. 13 shows a perspective view of the tool 1200 of FIG. 12. FIG. 13 shows more detail of the sides of the central disc of tool 1220 and of the cylindrical projections 1202, 1204, 1206, 1208, 1210, 1212, 1214 and 1216. Note that the diameter of each of the cylindrical projections 1202, 1204, 1206, 1208, 1210, 1212, 1214 and 1216 is generally the same diameter as the desired inner diameter of the plurality of loops located at the first and second ends 102 and 104 of the stent 100.

FIG. 14 shows a perspective view of the tool 1200 of FIG. 12, as it is used during the process of making the second end 104 of the stent 100. A plurality of wire strands have been bent and looped (such as wire strands 502, 702, 704, 706 of FIG. 7) using the cylindrical projections 1202, 1204, 1206, 1208, 1210, 1212, 1214 and 1216. That is, the cylindrical projections of the tool 1200 are used to form loops (such as 602, 712, 714 and 716 of FIG. 7) in the wire strands 502, 702, 704 and 706. Once the loops are formed, the wire strands 502, 702, 704 and 706 rest on the cylindrical projections 1202, 1204, 1206, 1208, 1210, 1212, 1214 and 1216. This provides a starting point for the braiding or weaving of the remaining portions of the stent 100. Subsequently, the remaining portions of the stent 100 are braided of woven using human or mechanical means.

It can be seen that a novel system has been disclosed in which a braided stent is formed with looped ends. Although illustrative embodiments of the invention have been shown and described, it is to be understood that various modifications and substitutions may be made by those skilled in the art without departing from the novel spirit and scope of the present invention. The modifications may be made which fall within the scope of the following claims.

What is claimed is:

1. An implantable stent, comprising:
a plurality of wire strands braided into a tubular structure such that the ends of the plurality of wire strands are located at a first end of the tubular structure; and
a first plurality of loops located at the first end of the tubular structure, each loop formed from a pair of ends of the plurality of wire strands.

2. The stent of claim 1, further comprising:
a second plurality of loops located at a second end of the tubular structure, each loop formed from a length of the plurality of wire strands.

3. The stent of claim 2, wherein the ends of the plurality of wire strands at the first end of the tubular structure are trimmed to be flush with the first plurality of loops.

4. The stent of claim 3, wherein the pair of ends of the plurality of wire strands that comprise each loop of the first plurality of loops are secured to each other.

5. The stent of claim 3, wherein the stent comprises the plurality of wire strands braided into axially spaced apart helices concentric on a central axis of the stent.

6. An implantable stent, comprising:
a plurality of wire strands braided into a tubular structure such that the ends of the plurality of wire strands are located at a first end of the tubular structure; and
a first plurality of loops located at a second end of the tubular structure, each loop formed from a length of the plurality of wire strands.

7. The stent of claim 6, further comprising:
a second plurality of loops located at the first end of the tubular structure, each loop formed from a pair of ends of the plurality of wire strands.

8. The stent of claim 7, wherein the ends of the plurality of wire strands at the first end of the tubular structure are trimmed to be flush with the second plurality of loops.

9. The stent of claim 8, wherein the pair of ends of the plurality of wire strands that comprise each loop of the first plurality of loops are secured to each other.

10. The stent of claim 8, wherein the stent comprises the plurality of wire strands braided into axially spaced apart helices concentric on a central axis of the stent.

11. A method for making an implantable stent, comprising:
forming a first plurality of loops from a plurality of wire strands;
arranging the first plurality of loops in a circle such that each loop is equally spaced apart around the circumference of the circle, wherein ends of the plurality of wire strands extend in the same axial direction perpendicular to the plane of the circle; and
braiding the plurality of wire strands to form a tubular structure, wherein the ends of the plurality of wire strands are located at a first end of the tubular structure.

12. The method of claim 11, further comprising:
forming a second plurality of loops at the first end of the tubular structure, each of the second plurality of loops formed from a pair of ends of the plurality of wire strands.

13. The method of claim 12, wherein the ends of the plurality of wire strands are trimmed to be flush with the second plurality of loops.

14. The method of claim 13, wherein the pair of ends that comprise each of the second plurality of loops are secured together.

15. The method of claim 13, wherein the braiding step further comprises:
braiding the plurality of wire strands into axially spaced apart helices concentric on a central axis of the tubular structure.

16. A method for making an implantable stent, comprising:
braiding a plurality of wire strands to form a tubular structure; and
forming a first plurality of loops at a first end of the tubular structure, each of the first plurality of loops formed from a pair of ends of the plurality of wire strands.

17. The method of claim 16, further comprising steps before the braiding step of:
forming a second plurality of loops from a plurality of wire strands;
arranging the second plurality of loops in a circle such that each loop is equally spaced apart around the circumference of the circle, wherein ends of the plurality of wire strands extend in the same axial direction perpendicular to the plane of the circle.

18. The method of claim 17, wherein the ends of the plurality of wire strands are trimmed to be flush with the first plurality of loops.

19. The method of claim 18, wherein the pair of ends that comprise each of the first plurality of loops are secured together.

20. The method of claim 18, wherein the braiding step further comprises:
braiding the plurality of wire strands into axially spaced apart helices concentric on a central axis of the tubular structure.
